(19) United States
(12) Patent Application Publication
(10) Pub. No.: US 2010/0166878 A1
(43) Pub. Date: Jul. 1, 2010

(54) COMPOSITION COMPRISING BEE VENOM FOR THE TREATMENT OF ATHEROSCLEROSIS

(76) Inventors: KWAN-KYU PARK, SUSEONG-GU (KR); JI-HYUN PARK, SUSEONG-GU (KR); KYUNG-HYUN KIM, SUSEONG-GU (KR); SOO-JUNG KIM, DALSEO-GU (KR); WOO-RAM LEE, CHANGNYEONG-GUN (KR); KWANG GILL LEE, SUWON-SI (KR); SANG MI HAN, SEOSAN-SI (KR); YOUNG-CHAE CHANG, SUSEONG-GU (KR); JUNG-YOON CHOE, SUSEONG-GU (KR); KEE-SIK KIM, SUSEONG-GU (KR)

Correspondence Address: FROMMER LAWRENCE & HAUG LLP, 745 FIFTH AVENUE, NEW YORK, NY, 10151, US

(21) Appl. No.: 12/613,330

(22) Filed: Nov. 5, 2009

(30) Foreign Application Priority Data
Dec. 26, 2008 (KR) 10-2008-0134621

(51) Int. Cl. A61K 35/64; A61P 9/10

(52) U.S. Cl. 424/539

(57) ABSTRACT

The present invention provides bee venom which can decrease expression levels of sclerotic factors, inflammatory factors and vascular adhesion factors associated with atherosclerosis, and a pharmaceutical composition comprising the bee venom as an active ingredient for the treatment of the atherosclerosis. When the bee venom was administered to laboratory animal models of atherosclerosis, the total cholesterol and neutral lipid were decreased, high-density cholesterol was maintained or even increased, the expression levels of TNF-α and IL-β as inflammation-associated cytokines were decreased in the blood, the expression levels of fibrosis-associated cytokines and vascular adhesion factors were decreased in the main artery and the heart, the plaque deposition generally caused by the atherosclerosis was decreased, and the expression levels of intercellular adhesion molecules (ICAM) and vascular cell adhesion molecules (VCAM), TGF-β1 and fibronectin as fibrosis-related cytokines were decreased.

### CROSS-REFERENCE TO RELATED APPLICATION

This application claims the benefit of Korean Patent Application No. 10-2008-0134622, filed on Dec. 26, 2008, the entire disclosure of which is hereby incorporated by reference.

### BACKGROUND OF THE INVENTION

1. Field of the Invention

The present invention relates to a composition comprising bee venom for the treatment of atherosclerosis, and more particularly, to a pharmaceutical composition comprising bee venom as an active ingredient for the treatment of atherosclerosis, the bee venom capable of decreasing expression levels of sclerotic factors, inflammatory factors and vascular adhesion factors involved in atherosclerosis.

2. Description of Related Art

Drastic changes in people's living conditions and eating habits has brought a considerable change in the types of diseases. Particularly, circulatory system diseases have become one of the major types of death-causing diseases in Korea as in the USA and Europe.

In Korea, the number of patients having various adult diseases and cardiovascular system diseases, such as hypertension, ischemic stroke, obesity, and diabetes, which are accompanied with atherosclerosis, has been continuously increasing due to the improvement in living standard and Westernized dietary habits, thereby considerably increasing the mortality therefrom.

Risk factors which induce atherosclerosis include hyperlipidemia, hypertension, smoking, diabetes, hyperuricemia, obesity, lack of exercise, crapulence. Of them, hypertension, smoking and diabetes are known as common risk factors in Korea, and recently hyperlipidemia has become the major risk factor. Due to the prevalence and the mortality due to atherosclerosis and the concomitant cardiovascular system diseases are increasing in the country, there has been an urgent need for the development of a method for the prevention and treatment of hyperlipidemia.

Currently, the “response-to-injury” hypothesis is the one attaining most support regarding the atherosclerosis incidence. According to the hypothesis, atherosclerosis is caused by damage and proliferation of artery endotheliocytes, movement of blood monocytes in the walls of blood vessels and transformation into macrophages, deposition of lipids and dead cell pieces, proliferation and fibrosis of smooth muscle cells, vasoconstriction and hemadostenosis caused by platelet adhesion and aggregation, etc., thereby thickening and narrowing blood vessels, and hardening the artery wall.

Until two decades ago, atherosclerosis had been merely considered as a degenerative process naturally occurring in old people with aging process. However, epidemiological investigation conducted over a few decades has revealed that various kinds of risk factors are somehow associated with an atherosclerosis lesion and that many kinds of cells and various growth factors and cytokines secreted by the cells are involved in a very complicated process leading to the lesion.

Various kinds of folk remedies or dietotherapies have been used in Korea to prevent atherosclerosis. In particular, herb medicines or natural products or their extracts have been widely used among people in the eastern part of Korea.

Bee venom, which is known to contain about 40 different substances stored in the venom sac, has been allegedly used to treat human diseases since the years before Christ. The bee venom has been actively studied in many countries including the U.S., China, Russia, and Northern European countries. Studies have been reported regarding modus operandi of venom components, irritability and toxicity of bee venom, immunotherapy, and treatments of arthritis, herpes simplex, multiple sclerosis, tumor, etc., However, they are mostly the ones transmitted among people, and not many clinical or fundamental and scientific studies have been performed.

Conventional technologies using bee venom relate to methods for the treatment of animals, such as a method for the treatment of arthritis of pigs using a raw bee venom (Korean Patent Application No. 10-1999-0005472), a method for the treatment of mastitis of pigs using a raw bee venom (Korean Patent Application No. 10-1999-0005477), a method for the treatment of agalactia of sow pigs using a raw bee venom (Korean Patent Application No. 10-1999-0005474), a method for the treatment of diarrhea of pigs using a raw bee venom (Korean Patent Application No. 10-1999-0005475), a method for the treatment of mastitis-metritis-agalactia (MMA) syndrome of sow pigs using a raw bee venom (Korean Patent Application No. 10-2000-0046411), a method for the treatment of diarrhea of calves using a raw bee venom (Korean Patent Application No. 10-2000-0046412), and a method for the treatment of endometritis of milk cows using a raw bee venom (Korean Patent Application No. 10-2001-0071220).

Moreover, Korean Patent No. 10-0668229 titled “Nuclear factor-Kappa B (NF-κB) inhibitor” discloses that bee venom and melittin as its principal component inhibit activation of NF-κB and control the DNA binding capacity, and thus the bee venom can be used as an NF-κB inhibitor or a transcription repressor controlling NF-κB involved DNA transcription. In addition, Korean Patent No. 10-0483496 titled “Antiinflammatory and analgesic composition comprising an aqueous fraction of bee venom” discloses bee venom fraction which produces antinociceptive and anti-inflammatory effects, the bee venom fraction comprising a substance below molecular weight of 10 KDa obtained from a water-soluble fraction prepared by removing hexane soluble substances from the bee venom through hexane extraction and removing ethyl acetate soluble substances therefrom through ethyl acetate extraction.

Additionally, Korean Patent No. 10-0844886 titled “Compositions for the treatment of vitiligo comprising bee venom and method for screening inhibitors against the pigmentation induced by bee venom” discloses a method for screening pigmentation inhibiting substances induced by a pharmaceutical composition for easily treating vitiligo and bee venom through high-throughput.

However, there had been no study conducted on the treatment of atherosclerosis using bee venom. Accordingly, the inventors of the present invention, after repeated studies on the efficacy of the bee venom, have established animal models of atherosclerosis induced by diet, to which bee venom was administered, to examine the influences of bee venom exerted on sclerotic factors, inflammatory factors and vascular adhesion factors associated with the atherosclerosis, and completed the preset invention.

### SUMMARY OF THE INVENTION

The present invention has been made in an effort to solve the above-described problems associated with the prior art, and an object of the present invention is to provide a pharmaceutical composition comprising bee venom as an active ingredient for the treatment of atherosclerosis.

The above object of the present invention has been achieved by inducing atherosclerosis to a laboratory animal by supplying high-cholesterol feed, dosing bee venom to the laboratory animal, separating the plasma from the laboratory animal, analyzing an expression level of inflammatory cytokine, measuring cholesterol in the serum of the laboratory animal, conducting protein separation and western blot analysis in the artery, heart and liver cell, and confirming the effects of the bee venom via biopsy.

In an embodiment, the present invention provides a pharmaceutical composition comprising bee venom as an active ingredient for the treatment of atherosclerosis.

According to the present invention, the “active ingredient” represents a substance or a substance group which is expected to directly or indirectly express the potency and efficacy of the medical supplies concerned by its intrinsic pharmaceutical action (including herb medicines and the likes comprising unrevealed pharmaceutically active components) as a major component.

Moreover, the pharmaceutical composition may comprise a mistletoe extract and other carriers or excipients and may be provided in the form of tablets, pills, granules, liquid, capsules, etc., which are obvious to those skilled in the art.

In the experiment using animal models with atherosclerosis, the bee venom according to the present invention was shown to decrease the total cholesterol and neutral lipid but maintain or even increase the high-density cholesterol.

In one embodiment of the invention, the total cholesterol level associated with treatment with bee venom to a patient in need thereof decreases by about 10-25% relative to a treatment without bee venom.

In another embodiment of the invention, triglyceride levels associated with treatment with bee venom to a patient in need thereof decreases by about 0-50% relative to a treatment without bee venom. In another embodiment regarding triglyceride levels, the triglyceride level is maintained at a level of about 80-125% of the initial triglyceride level at the start of treatment.

In another embodiment of the invention, HDL (high density cholesterol levels) levels associated with treatment with bee venom to a patient in need thereof increases by about 10-25% relative to a treatment without bee venom. In another embodiment regarding HDL levels, HDL increases by about 30 to 60% relative to the initial HDL at the start of treatment.

Further, in the experiment using the animal models with atherosclerosis, the bee venom according to the present invention was shown to decrease the expression levels of TNF-α and IL-β as inflammation-associated cytokines in the blood of the above experimental animals; fibrosis-associated cytokines and vascular adhesion factors in the main artery of experimental animals; and plaque deposition generally caused by atherosclerosis.

In addition, the bee venom according to the present invention decreased expression levels of intercellular adhesion molecules (ICAM) and vascular cell adhesion molecules (VCAM) as vascular adhesion factors, and those of TGF-β1 and fibronectin as fibrosis-related cytokines, and fibrosis-associated cytokines and vascular adhesion factors in the heart.

When the bee venom according to the present invention was administered to the laboratory animal models of atherosclerosis, the amount of the total cholesterol and neutral lipid was decreased, respectively; the level of the high-density cholesterol was maintained or increased; the expression levels of TNF-α and IL-β, which are inflammation-associated cytokines, were decreased in the blood; the expression levels of fibrosis-associated cytokines and vascular adhesion factors were decreased in the main artery and the heart; the plaque deposition generally caused by the atherosclerosis was decreased, and the expression levels of ICAM and VCAM which were vascular adhesion factors, TGF-β1 as fibrosis cytokine and fibronectin were decreased. Therefore, the bee venom according to the present invention is very effective for the treatment of atherosclerosis.

### BRIEF DESCRIPTION OF THE DRAWINGS

The above and other features of the present invention will be described with reference to certain exemplary embodiments thereof illustrated the attached drawings in which:

FIGS. 1A to 1C are graphs illustrating the influences of bee venom exerted on cholesterol in animal models of atherosclerosis, wherein A represents the total cholesterol, B represents the neutral lipid, and C represents the HDL-cholesterol;

FIGS. 2A and 2B are graphs illustrating the influences of bee venom exerted on inflammation-associated cytokine in animal models of atherosclerosis, wherein A represents the productivity of TNF-α and B represents the productivity of IL-β;

FIGS. 3A to 3C are views illustrating influences of bee venom exerted on fibrosis and vascular adhesion factors in the main artery ablated from animal models of atherosclerosis, wherein A represents the H&E staining, B represents the confirmation at the protein level expression, and C represents the immunohistochemical staining; and

FIGS. 4A and 4B are views illustrating influences of bee venom exerted on fibrosis and vascular adhesion factors in the heart ablated from animal models of atherosclerosis, wherein A represents the H&E staining, B represents the confirmation at the protein level expression, and C represents the immunohistochemical staining.

### DETAILED DESCRIPTION OF THE INVENTION

Hereinafter, preferred embodiments of the present invention will be described in detail with reference to the accompanying drawings. However, it is to be noted that the scope of the present invention is not limited to the following embodiments.

### EXAMPLES

In the exemplary embodiments of the present invention, 10 week-old C57BL/6 rats (20 to 25 g) were purchased from Samtako, Inc. and used as laboratory animals after an acclimation period of one week. LPS (2 mg/kg) was injected into an abdominal cavity of the laboratory animal models three times to induce atherosclerosis. Moreover, as high-cholesterol feed for inducing atherosclerosis, 15% fat, 1.25% cholesterol and 0.5% cholic acid were mixed with normal diet powder feed and prepared in the form of pellets. It was fed ad libitum for 12 weeks.

Bee venom, which was supplied by the National Academy of Agricultural Science of the Rural Development Administration in Korea, was injected into the abdominal cavity of the laboratory animal at a concentration of 0.1 mg/kg twice a week for 12 weeks.

Experimental groups were divided into an experimental group

(LPS+Fat) of atherosclerosis, an experimental group (LPS+Fat+BV) of atherosclerosis to which bee venom (0.1 mg/kg) was applied, and a normal control (NC) group. The laboratory animals were sacrificed after 2 weeks, 4 weeks, 8 weeks, and 12 weeks, respectively. The blood was extracted from the heart, and the tissue was ablated from the main artery and fixed to 10% buffered neutral formalin solution.

Experiment analysis results were described using the mean and the standard deviation (mean±S.D.) with respect to each group, and all data were processed using a statistical package for social science (SPSS) program (SPSS Inc., Chicago, Ill., USA) and confirmed at a significance level of p<0.05 adopting analysis of variance (ANOVA) by repeated measurements.

### Example 1

### Confirmation of Expression Levels of Inflammatory Cytokines (TNF-α and IL-1β)

The expression levels of inflammatory cytokines were measured in the plasma separated from the laboratory animals using an enzyme-linked immunosorbent assay (ELISA) kit (R&D, Minneapolis, Minn.). Primary antibodies and secondary antibodies of TNF-α and IL-1β were reacted in order and color-developed with 3,3′,5,5′-tetramethylbenzidine. Then, the optical density was measured at 450 nm by using an ELISA reader. The concentration of the sample was converted into standard units using standard solutions of TNF-α and IL-1β.

### Example 2

### Analysis of Cholesterol

Total cholesterol, triglyceride and high-density lipoprotein (HDL)-cholesterol were measured in the serum separated from the laboratory animals. Cholesterol analysis reagents were supplied from each kit of Asan pharmaceutical Co. Ltd., the standard units were described using each standard solution, and the concentration of the sample was converted by the graph.

### Example 3

### Protein Separation and Western Slot Analysis

The artery, heart and liver cell were added with IPH elution buffer (50 mM pH 8.0 Tris, 150 mM NaCl, 5 mM EDTA, 0.5% NP-40, 100 mM PMSF, 1 mg/mL leupeptin, 1 mg/mL aprotinin and 1 M DTT), reacted at 4° C. for 30 min, and centrifuged at 12,000 rpm for 10 min. Protein samples were separated and quantitated by Bradford method (Bio-Rad Laboratories, CA, USA), electrophoresed through sodium dodecyl sulfate-polyacrylamide gel electrophoresis (SDS-PAGE), and transferred into a polyvinylidene fluoride (PVDF) membrane (Milipore, USA). The membrane was reacted with anti-TGF-α, anti-α-SMA, anti-fibronectin, anti-ICAM-1 and anti-VCAM-1 as primary antibodies, washed twice for 15 min with tris buffered saline tween 20(TBS-T) and reacted with secondary antibodies for 2 hours. The expression analysis of the antibodies was conducted using an enhanced chemiluminescence Western blot analysis system (Amersham, NJ, USA) expressed by horseradish peroxidase (HRP)-linked secondary antibodies, which were developed on X-ray film and analyzed using Science Lab 2005 (Fujifilm, Japan).

### Example 4

### Incontinentia Examination

Immunohistochemical staining was performed to examine expression inhibition of TGF-β1, α-SMA, anti-ICAM and anti-VCAM in the tissue. First, a biopsy tissue of the kidney embedded with paraffin was cut into slices of a thickness of 4 μm by a rotation microtome, attached to a bonding agent-processed slide, deparaffinized, and hydrated using ethanol and distilled water. Dehydrated tissue section was incubated with 3% hydrogen peroxide solution diluted with methanol for 30 min to prevent reaction with endogenous peroxidase and washed with distilled water. The resulting tissue section was immersed in a 95° C. dako epitope retrieval solution (0.01 mol/L citrate buffer, pH 6.0) which was previously heated, boiled for 10 min to perform epitope retrieval, cooled at room temperature, and washed with a phosphate buffered solution (PBS). Primary antibodies against TGF-β1, α-SMA, anti-fibronectin, anti-ICAM and anti-VCAM were reacted in the tissue at 4° C. for 16 hours, reacted with biotinylated anti-mouse immunoglobulin G (IgG) (DAKO, CA, USA) at 37 (C for 15 min., reacted with sepoavidin peroxidase (DAKO, CA, USA) at 37 (C for 15 min., color-developed with 3,3′-diaminobezidine tetrahydrochloride (DAB), contrast-stained with hemotoxylin, and observed under light microscope.

### Example 5

### Test Results

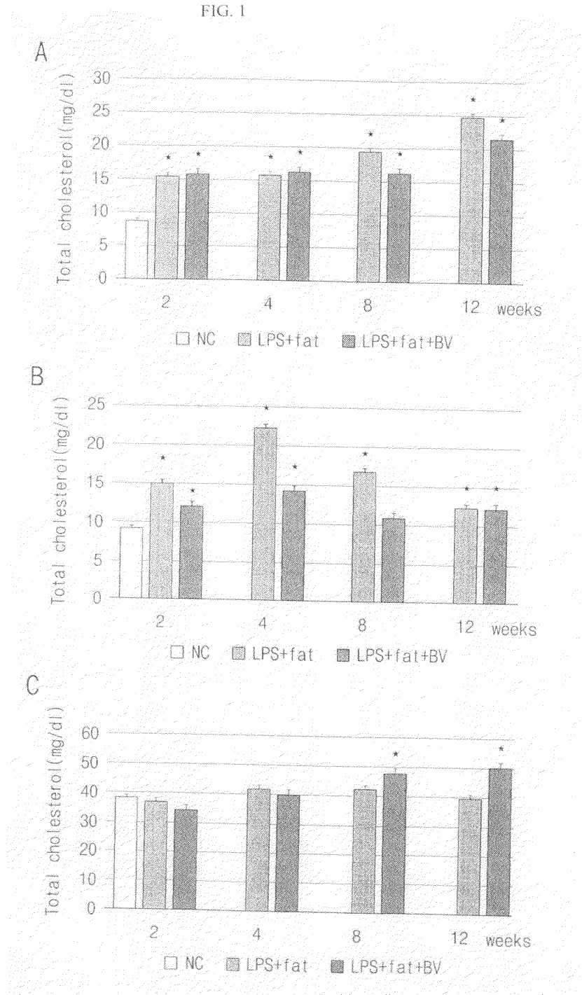

As illustrated in FIG. 1, after the bee venom was administered to the animal models of atherosclerosis, the amounts of total cholesterol, neutral lipid and high-density cholesterol were measured in the extracted blood. As a result, the total cholesterol and the neutral lipid were decreased in the experimental group administered with the bee venom after 2 weeks, 4 weeks, 8 weeks and 12 weeks, respectively, and the high-density cholesterol was maintained or increased therein, thus demonstrating the effects of the bee venom. The high-density cholesterol was maintained or increased therein, thus demonstrating the effects of the bee venom.

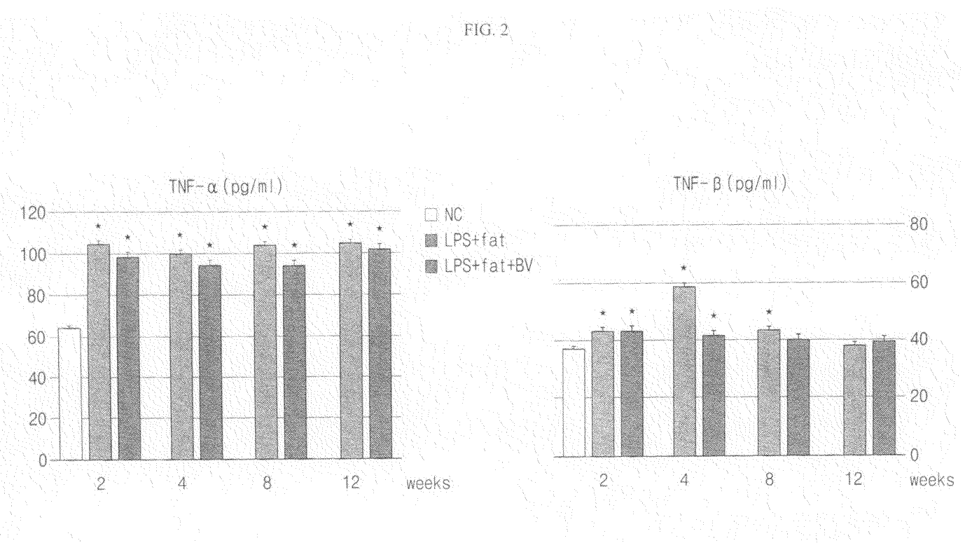

Moreover, as illustrated in FIG. 2, after the bee venom was administered to the animal models of atherosclerosis, TNF-α and IL-β as inflammation-associated cytokines were measured in the extracted blood by the ELISA. As a result, they were decreased in the experimental group administered with the bee venom after 2 weeks, 4 weeks, 8 weeks and 12 weeks, thus demonstrating the effects of the bee venom.

After the bee venom was administered to the animal models of atherosclerosis, fibrosis-associated cytokines and vascular adhesion factors were measured in the main artery extracted from the tissue protein. As a result, they were decreased after 2 weeks, 4 weeks, 8 weeks and 12 weeks, thus demonstrating the effects of the bee venom.

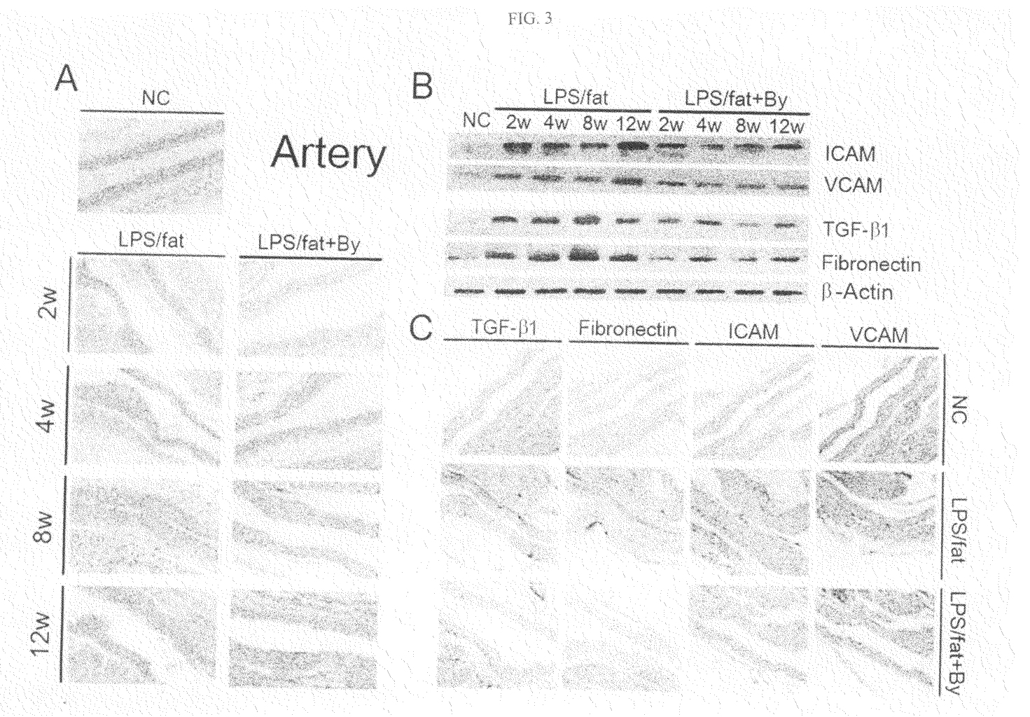

When the tissue was observed by H&E staining, the plaque deposition generally caused by the atherosclerosis occurred in the atherosclerosis from 2 to 12 weeks. However, it was confirmed that the plaque deposition was reduced in the experimental group administered with the bee venom (refer to FIG. 3A).

The expression levels of intercellular adhesion molecules (ICAM) and vascular cell adhesion molecules (VCAM) as the vascular adhesion factors, TGF-(1 and fibronectin as fibrosis-related cytokines using western blot analysis were reduced in the experimental group administered with the bee venom (refer to FIG. 3B). In addition, the result using immunohistochemical staining was the same as the Western blot analysis.

After the bee venom was administered to the animal models of atherosclerosis, the fibrosis-associated cytokines and vascular adhesion factors were measured in the extracted heart at tissue and tissue protein. As a result, they were all decreased after 2 weeks, 4 weeks, 8 weeks and 12 weeks, thus demonstrating the effects of the bee venom.

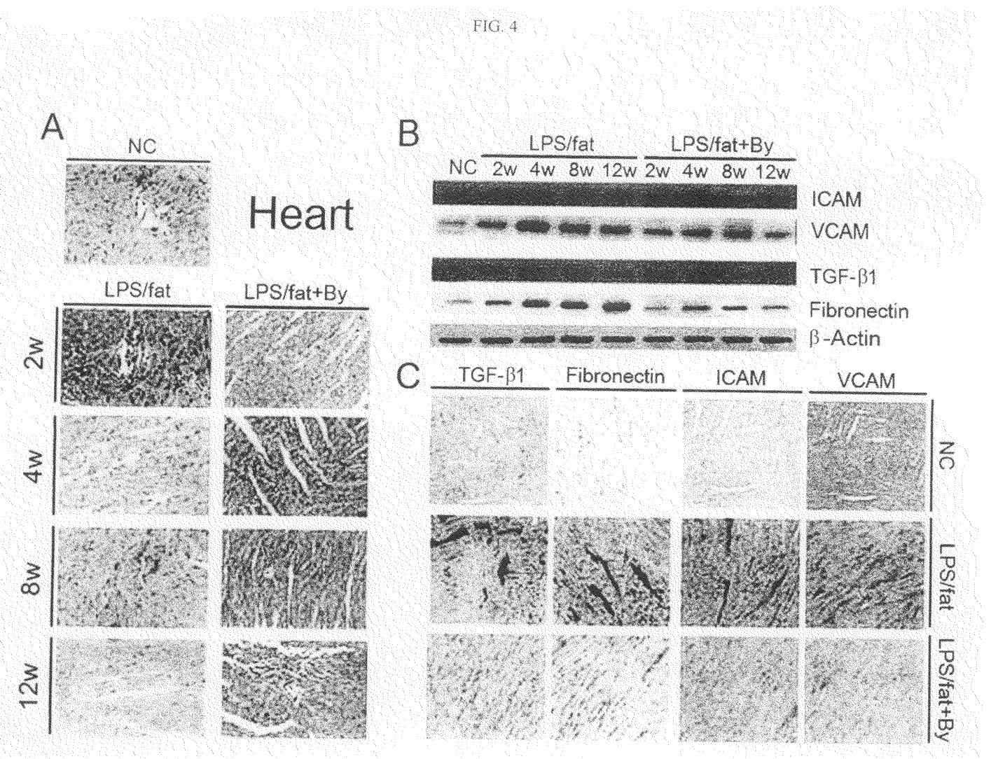

When the tissue was observed by H&E staining, the plaque deposition generally caused by the atherosclerosis occurred in the atherosclerosis from 2 to 12 weeks. However, it was confirmed that the plaque deposition was reduced in the experimental group administered with the bee venom (FIG. 4A).

The expression levels of intercellular adhesion molecules (ICAM) and vascular cell adhesion molecules (VCAM) as the vascular adhesion factors,

TGF-β1 and fibronectin as fibrosis-related cytokines using western blot analysis were reduced in the experimental group administered with the bee venom (refer to FIG. 3B). In addition, the result using immunohistochemical staining was the same as the Western blot analysis.

As shown above, preferred embodiments of the present invention have been described and illustrated, however, the present invention is not limited thereto, rather, it should be understood that various modifications and variations of the present invention can be made thereto by those skilled in the art without departing from the spirit and the technical scope of the present invention as defined by the appended claims.

1. A method of treating atherosclerosis which comprises of administering an effective amount of the bee venom to a patient in need thereof.

2. The method of claim 2, wherein the bee venom is administered via injection.
